(19) United States
(12) Patent Application Publication
(10) Pub. No.: US 2006/0115536 A1
(43) Pub. Date: Jun. 1, 2006

(54) Glycerin based synthesis of silver nanoparticles and nanowires

(75) Inventors: Miguel Jose Yacaman, Lakeway, TX (US); Jose Luis Elechiguerra, Austin, TX (US); Justin Lockheart Burt, Austin, TX (US); Jose Ruben Morones, Austin, TX (US); Leticia Larios Lopez, Austin, TX (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/271,083

(22) Filed: Nov. 11, 2005

Related U.S. Application Data
(60) Provisional application No. 60/627,372, filed on Nov. 12, 2004.
(60) Provisional application No. 60/627,987, filed on Nov. 15, 2004.

(51) Int. Cl. A61K 33/38; A61K 9/14

(52) U.S. Cl. 424/489; 424/618; 977/915

(57) ABSTRACT

The present invention includes compositions and methods for the production of noble metal nanoparticles and nanowires conjugated to polyols or polymers. The present invention allows the incorporation of noble metal nanoparticles to a wide range of products such as body care products to exploit the biocidal properties of silver nanoparticles against bacteria, viruses and fungi.

This application claims priority to U.S. Provisional Patent Application Ser. No. 60/627,372, filed Nov. 12, 2004 and U.S. Provisional Patent Application Ser. No. 60/627,987, filed Nov. 15, 2004, the entire contents of which are incorporated herein by reference. Without limiting the scope of the invention, its background is described in connection with nanoparticles.

### TECHNICAL FIELD OF THE INVENTION

The present invention relates in general to the field of antivirals, and more particularly, to compositions, methods and treatment of viral particles with silver nanoparticles to reduce or eliminate viral infection and/or transmission.

### BACKGROUND OF THE INVENTION

Nanowires, as one dimensional nanostructured materials, have become the focus of intensive research owing to their great potential for use as building blocks in the fabrication of electronic, optoelectronic and sensor devices with nanoscale dimensions. The most common applications of nanowires are expected to be in electromagnetic and energy storage devices.

Silver (Ag) has many important applications due to its high electrical and thermal conductivity and its unique optical properties that depend on size and shape. Therefore, the study of Ag nanowires has led to great interest.

Glycerin is also used in many body care products. U.S. Pat. No. 6,720,006, teaches a body care product is a product that is brought into contact with human and/or animal skin or mucosa to provide a cleaning, protective, therapeutic, cosmetic or soothing benefit. These products can also be found on surfaces contacting the skin such as diapers, incontinence articles, catamenial devices, training pants, panty liners, etc.; or skin care compositions such as emulsions, lotions, creams, ointments, salves, powders, suspensions, gels, soaps, etc.

### SUMMARY OF THE INVENTION

In response to the growing threat of AIDS transmission, the use of condoms during sexual intercourse has been suggested as a means of preventing transmission of the AIDS virus. Improper use of condoms, or their perforation during intercourse, renders them only partially effective. Accordingly, there is a pressing need for a better method of inhibiting the transmission of the AIDS virus in humans during sexual intercourse and during surgical procedures on infected patients. The present invention provides compositions and methods for making and using an anti-viral composition for use in treating and preventing viral infection.

The present invention includes compositions, methods of making and methods of using silver nanoparticles. More particularly, it includes the synthesis of silver nanoparticles (particles of sizes between 1 and 100 nm) and nanowires (1-D structures with diameters between 1 and 100 nm with lengths up to several hundreds of nanometers) using glycerin as both the reducing agent and the solvent of the nanostructures. However, this technique may be extended but not limited to nanoparticles and nanowires of gold, platinum, palladium, copper, iron, and alloys composed of these metals. It can be also extended to metal oxides nanoparticles and nanowires such as titanium dioxide, zirconium dioxide, etc. It is important to mention that the method can be also expanded to the production of particles in the mesoscopic range, specifically from 100 to 500 nm. Several capping agents can be used, e.g. polyvinylpyrrolidone (PVP).

A current problem is the resistance developed by bacteria to current antibiotics. In addition, there are no 100% efficient treatments and vaccines to prevent or combat diseases due to viruses such as HIV, hepatitis C (HCV), human papillomavirus (HPV), etc. Due to its strong toxicity to a wide range of microorganisms, silver has been used against bacteria and fungi. There is a possibility of using nanotechnology to improve and develop silver nanoparticles to use as a biocide in substitution of current products like antibiotics. In fact, it is disclosed herein that the properties of silver nanoparticles in different forms are able to deactivate HIV with concentrations below the cytotoxic concentrations for MT2 cells.

The chemical and physical properties that bulk materials exhibit change drastically when the material is in the nanometer range. For this reason there is an increasing appeal in the development of nanomaterials, which can be used in physical, biological, biomedical and pharmaceutical applications.

The fact that glycerin is the solvent for the nanoparticles and/or nanowires allows these structures to be used in almost any current commercial application of glycerin, such as preservation of fruit, prevention of freezing in hydraulic jacks, lubrication for molds, some printing inks, cake and candy making, and as an antiseptic. The present technology offers the possibility of combining the biocidal properties of silver nanoparticles with the versatile properties of glycerin in body care products. However, the present invention is not limited to body care products. Glycerin is miscible with water so other applications, such as paints, plastics and other composite materials can be implemented.

The present invention includes the synthesis and characterization of silver nanowires synthesized by the polyol method. In an alternative method, ethyleneglycol (EG) may be used as both reducing reagent and solvent. Poly(vinylpyrrolidone) (PVP) plays a role of structure-directing agent or capping agent. Nanowires were also synthesized by a modified polyol method using glycerin (G) instead of EG and poly(diallyldimethyl ammonium chloride) (PDDAM) replacing PVP.

### BRIEF DESCRIPTION OF THE DRAWINGS

For a more complete understanding of the features and advantages of the present invention, reference is now made to the detailed description of the invention along with the accompanying figures and in which:

FIG. 1a is an SEM image of the synthesized silver nanowires. The faceting in the nanowires is clearly observed. The inset shows a lower magnification SEM image of the same sample. FIG. 1b is a schematic model of the nanowires. FIG. 1c is an XRD pattern of the same sample. FIG. 1d is an EDS spectrum of one nanowire. The C signal comes from both the TEM grid and the PVP coating of the nanowires; O and N are also from the PVP coating, while Cu comes from the TEM grid.

FIG. 2a is an X-ray photoelectron spectra of pure PVP. FIG. 2a is the PVP repeating unit, the three different carbon species are labeled as 1, 2, and 3. FIG. 2b is a C 1s spectrum. FIG. 2c is an N 1s spectrum. FIG. 2d is an O 1 s spectrum.

FIG. 3a to 3d are X-ray photoelectron spectra of PVP-coated silver nanowires. FIG. 3a is C 1s spectrum. FIG. 3b is an N 1s spectrum. FIG. 3c is a O 1s spectrum. FIG. 3d is Ag 3d_{5/2} and Ag 3d_{3/2} spectra.

FIGS. 4a to 4f are TEM images of the same sample at different times after exposure to air at ambient conditions. FIG. 4a is a sample just after synthesis. FIGS. 4b and FIG. 4c are images of the sample after 3 weeks. FIGS. 4d-FIG. 4f are images after 4, 5, and 24 weeks, respectively.

FIG. 5a to 5d are SEM images of silver nanowires at different times after exposure to air at ambient conditions. FIG. 5a and FIG. 5b are images of the sample after 4 weeks. FIG. 5c and FIG. 5d are SEM images of the sample presented in FIG. 4f.

FIGS. 6a and 6b are TEM images of two different samples that were not exposed to periodical electron irradiation. Sample after (FIG. 6a) 6 weeks and (FIG. 6b) 24 weeks.

FIG. 7 are HAADF images at different magnifications of the nanowire shown in FIG. 6b.

FIGS. 8a to 8b are a compositional analysis of one of the tips of the nanowire presented in the previous figure. FIG. 8b is an EDS line scan across the shell of crystallites. FIG. 8c is an EDS line scan across the core region of the nanowire. (FIG. 8d) Punctual EDS analysis of three different regions of the tip of the wire.

FIGS. 9a and 9b are high-magnification TEM image of the body of one nanowire after 24 weeks of exposure to air at ambient conditions. FIG. 9b is a high-resolution TEM image of one of the crystallites that compose the shell. The inset corresponds to the FFT of the image.

FIG. 10 includes electron microscopy images of different regions of the sample after 24 weeks of exposure to air.

FIGS. 11a to 11s are SEM image of the sample of silver nanowires after sulfidation. FIG. 11b to FIG. 11d are HAADF images of the sulfidized silver nanowires.

FIGS. 12a to 12d are EDS mapping of the sulfidized nanowire presented in FIG. 12a.

FIG. 12b is a silver map. FIG. 12c is a sulfur map. FIG. 12d is a carbon map. In the last panel, the lacey carbon grid is clearly observed.

FIGS. 13a and 13b are EDS punctual analysis of (FIG. 13b) two different regions in one of the tips of the sulfidized nanowires presented in (FIG. 13a).

FIGS. 14a to 14e are X-ray photoelectron spectra of sulfidized silver nanowires. FIG. 14a is a C 1s spectrum. FIG. 14b is an N 1s spectrum. FIG. 14c is an O 1s spectrum. FIG. 14d is an Ag 3d_{5/2} and Ag 3d_{3/2} spectra. FIG. 14e is an S 2p_{3/2} and S 2p_{1/2} spectra.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “antiviral” and “antiviral composition” refer to an amount of anti-viral protein associated noble metal nanoparticles treated with a polyol or polymer that suppress the replication and the spread of viruses, prevent viral attachment, prevent viral replication within the host cell, and/or improving or alleviating the symptoms caused by viral infection. One example of the present invention is a glycerol-treated silver nanoparticle, nanorod or nanotube. The criteria for effective therapy include lower viral load, lower mortality rate, and/or lower morbidity rate, etc.

As used herein, “derivatives” refers to any derivative of the polyol-treated noble metal nanoparticles or nanowires and combinations thereof. Non-limiting example of protein associated noble metal nanoparticles include nanoparticles that associate with one or more proteins via covalent or non-covalent bonding and may include combinations of proteins and even concatamers of protein-nanoparticle-protein, etc., into bi-, tri-, terta-, multimers, oligomers, polymers and the like in two or three-dimensions.

As used herein, “delivering” refers to contacting polyol-treated noble metal nanoparticles or nanowires to a location or target defined as effecting the placement of the nanoparticles attached to, next to, or sufficiently close to the location such that any heat generated by the nanoparticles is transferred to the location. “Delivering” may be targeted or non-targeted as the term “targeted” is used herein.

As used herein, “Nanometer” is 10⁻⁹ meter and is used interchangeably with its abbreviation “nm.”

As used herein, “nanoparticle” refers to defined as a noble-metal particle having dimensions of from 1 to 5000 nanometers, having any size, shape or morphology. For use with the present invention the nanoparticles are noble metals, such as gold colloid or silver and may be, e.g., nanospheres, nanotubes, nanorods, nanocones and the like.

As used herein, “nanoparticle” refers to one or more nanoparticles. As used herein, “nanoshell” means one or more nanoshells. As used herein, “shell” means one or more shells.

As used herein, “non-tissue” is defined as any material that is not human or animal tissue. As used herein, the term “targeted” refers to the use of protein-protein binding, protein-ligand biding, protein-receptor binding, and other chemical and/or biochemical binding interactions to direct the binding of a chemical species to a specific site.

As used herein, the term “polyol” refers to a compound, polymer or oligomer containing two or more hydroxyl groups. Furthermore, the polyol may have one or more hydroxyl groups supplied from a carboxylic acid. The polyols of the present invention may be aliphatic, aromatic, heteroaliphatic, saturated alicyclic, saturated heteroalicyclic, aromatic, heteroaromatic, or polymeric. The hydroxyl groups of the polyol may be located at the terminal groups or as groups that are pendant from the backbone chain. The molecular of the polyol can generally vary depending on the application and, e.g., if the polyol portion is a monomer, di-mer, tri-mer, oligomer, polymer, whether linear, branched and/or aromatic. General examples of polyols include glycerin, glycols, polyglycols and polyglycerols, polyethers and polyesters. The polyol refers to the attachment of such a moiety to the noble metal nanoparticles or nanowires described herein.

Representative examples of non-polymeric polyols include alkylene glycols (e.g., 1,2-ethanediol, 1,2-propanediol, 3-chloro-1,2-propanediol, 1,3-propanediol, 1,3-butanediol, 1,4-butanediol, 2-methyl-1,3-propanediol, 2,2-dimethyl-1,3-propanediol (neopentylglycol), 2-ethyl-1,3-propanediol, 2,2-diethyl-1,3-propanediol, 1,5-pentanediol, 2-ethyl-1,3-pentanediol, 2,2,4-trimethyl-1,3-pentanediol, 3-methyl-1,5-pentanediol, 1,2-, 1,5-, and 1,6-hexanediol, 2-ethyl- 1,6-hexanediol, bis(hydroxymethyl)cyclohexane, 1,8-octanediol, bicyclo-octanediol, 1,10-decanediol, tricyclo-decanediol, norbornanediol, and 1,18-dihydroxyoctadecane); polyhydroxyalkanes (e.g., glycerine, trimethylolethane, trimethylolpropane, 2-ethyl-2-(hydroxymethyl)-1,3-propanediol, 1,2,6-hexanetriol, pentaerythritol, quinitol, mannitol, and sorbitol); and other polyhydroxy compounds, e.g., diethylene glycol, triethylene glycol, tetraethylene glycol, tetramethylene glycol, dipropylene glycol, diisopropylene glycol, tripropylene glycol, 1,11-(3,6-dioxaundecane)diol, 1,14-(3,6,9,12-tetraoxatetradecane)diol, 1,8-(3,6-dioxa-2,5,8-trimethyloctane)diol, 1,14-(5,10-dioxatetradecane)diol, castor oil, 2-butyne-1,4-diol, N,N-bis(hydroxyethyl)benzamide, 4,4′-bis(hydroxymethyl)diphenylsulfone, 1,4-benzenedimethanol, 1,3-bis(2-hydroxyethyoxy)benzene, 1,2-, 1,3-, and 1,4-resorcinol, 1,6-, 2,6-, 2,5-, and 2,7-dihydroxynaphthalene, 2,2′- and 4,4′-biphenol, 1,8-dihydroxybiphenyl, 2,4-dihydroxy-6-methylpyrimidine, 4,6-dihydroxypyrimidine, 3,6-dihydroxypyridazine, bisphenol A, 4,4′-ethylidenebisphenol, 4,4′-isopropylidenebis(2,6-dimethylphenol), bis(4-hydroxyphenyl)methane, 1,1-bis(4-hydroxyphenyl)-1-phenylethane (bisphenol C), 1,4-bis(2-hydroxyethyl)piperazine, bis(4-hydroxyphenyl) ether, as well as other aliphatic, heteroaliphatic, saturated alicyclic, aromatic, saturated heteroalicyclic, and heteroaromatic polyols, combinations or mixtures thereof.

Representative examples of polymeric polyols include polyoxyethylene, polyoxypropylene and ethylene oxide-terminated polypropylene glycols and triols; polytetramethylene glycols; polydialkylsiloxane diols; hydroxy-terminated polyesters and hydroxy-terminated polylactones (e.g., polycaprolactone polyols); hydroxy-terminated polyalkadienes (e.g., hydroxyl-terminated polybutadienes); and the like. In addition mixtures of polymeric polyols can be used if desired.

Specific polyols include 1,2-ethanediol; 1,2- and 1,3-propanediol; 1,3- and 1,4-butanediol; neopentylglycol; 1,5-pentanediol; 3-methyl- 1,5-pentanediol; 1,2-, 1,5-, and 1,6-hexanediol; bis(hydroxymethyl)cyclohexane; 1,8-octanediol; 1,10-decanediol; di(ethylene glycol); tri(ethylene glycol); tetra(ethylene glycol); di(propylene glycol); di(isopropylene glycol); tri(propylene glycol); polyoxyethylene diols; polyoxypropylene diols; polycaprolactone diols; resorcinol; hydroquinone; 1,6-, 2,5-, 2,6-, and 2,7-dihydroxynaphthalene; 4,4′-biphenol; bisphenol A; bis(4-hydroxyphenyl)methane; and the like; and mixtures thereof. More preferred are 1,2-ethanediol; 1,2- and 1,3-propanediol; neopentylglycol; 1,2- and 1,6-hexanediol; di(ethylene glycol); poly[di(ethylene glycol) phthalate] diol; poly(ethylene glycol) diols; polydimethylsiloxane diol; polypropylene glycol; dimer diol; polycaprolactone diol; bisphenol A; resorcinol; hydroquinone; and mixtures thereof.

As used herein, “viral infection” refers to viral invasion of a target cell. When the virus enters the healthy cell, it takes advantage of the host reproduction mechanism to reproduce itself, ultimately killing the cell. As the virus reproduces, newly produced viral progeny infect other cells, often adjacent cells. Some viral genes can also integrate into host chromosome DNA to cause a latent infection via a provirus. The provirus reproduces itself with the replication of the host chromosome, and can bring the infected people into morbidity at any moment if activated by various factors inside and outside the body.

As used herein, “synergic action” refers to a joint protein associated polyol-treated noble metal nanoparticles or nanowires drug administration that is more effective than the additive action of merely using any of two or more therapeutics to cure or to prevent viral infection. The synergic effect can increase the efficacy of the antiviral drugs and the protein associated noble metal nanoparticles to avoid or alleviate viral tolerance against any single medicine.

As used herein, “therapeutics” refers the protein associated noble metal nanoparticles or nanowires whether alone or compounded in a delivery system, whether liquid, solid, gel-like, dried, frozen, resuspended and the like. The protein associated noble metal nanoparticles drug or active agent is conductive to the treatment of viral infection or virus-caused diseases, as taught herein.

The protein associated noble metal nanoparticle or nanowire antiviral agents of the present invention may be used alone or in combinations with agents that include, but are not limited to antiviral agents, such as the cytokines rIFNα, rIFNβ, and rIFNγ; reverse transcriptase inhibitors, such as AZT, 3TC, ddI, ddC, Nevirapine, Atevirdine, Delavirdine, PMEA, PMPA, Loviride, and other dideoxyribonucleosides or fluorodideoxyribonucleoside; viral protease inhibitors, such as Saquinavir, Ritonavir, Indinavir, Nelfinavir, and VX-478; hydroxyurea; viral mRNA capping inhibitors, such as viral ribovirin; amphotericin B; ester bond binding molecule castanospermine with anti-HIV activity; glycoprotein processing inhibitor; glycosidase inhibitors SC-48334 and MDL-28574; virus absorbent; CD4 receptor blocker; chemokine co-receptor inhibitor; neutralizing antibody; integrase inhibitors, and other fusion inhibitors.

The anti-viral protein-nanoparticles described herein may be used as part of a method and kit for improved antiviral therapy for the treatment of broad viral (including HIV) infection. In addition, the present invention provides a method of joint drug administration aimed at boosting the therapeutic effect, including the use of combination therapy, its derivatives, a second active agent or nutraceutical or dietary supplement, generally provided alone or in combination within a pharmacologically acceptable carrier. An advantage of combination therapy is that it may preclude viral adaptation or mutation that increases its tolerance against each therapeutic alone. Another advantage of combination therapy is that drugs may be provided at lower doses to reduce drug toxicity and enhance the therapeutic index.

It is known that size confinement produces dramatic changes on the physical properties of matter. One of the most well-known effects is the change of optical properties in noble metal nanoparticles with size, known generally as the surface Plasmon resonance effect. Noble metal nanoparticles or nanowires produce changes in the color, i.e., the light scattering by surface plasmons. In the case of transition metals the search for ultra dense magnetic recording devices has promoted the research in nanoparticles. Finite size can have effects on the structural and magnetic order in nanoparticles.

It is known that size confinement produces dramatic changes on the physical properties of matter. This has been the subject of nanotechnology studies for several years. Probably one of the most well-known effects is the change of optical properties in noble metal nanoparticles and nanowires with size. Noble metal nanoparticles and nanowires produce spectacular changes in the color, i.e., the light scattering by surface plasmons. In the case of transition metals the search for ultra dense magnetic recording devices has promoted the research in nanoparticles and nanowires. Finite size can have effects on the structural and magnetic order in nanoparticles and nanowires. It has been found that the present invention may be used alone or in a medium or carrier, where the silver nanoparticles are homogeneously dispersed, and is very friendly to the human body and can be rapidly incorporated to a variety of body care products to exploit the biocidal properties of silver nanoparticles against a wide range of toxic microorganisms (bacteria, viruses and fungi).

One important characteristic of the present invention is the use of glycerin as both the solvent and the reducing agent in the production of silver nanoparticles. Using glycerin and equivalent compounds as the solvent and reducing agent allows noble metal nanoparticles and nanowires, e.g., silver nanoparticles, to be dispersed well in a solvent that is extensively used in many applications, so the biocidal properties of silver nanoparticles can be exploited in many of those products. A well-established method found in the literature for the synthesis of metal and metal oxide nanoparticles and nanowires is known as the polyol method. In this technique, ethylene glycol is used as both the reducing agent and the solvent. The main difference in the proposed technology is the replacement of ethylene glycol by glycerin (propylene glycol) which is a friendlier and less toxic compound. The physical properties of both compounds are different, so the general behavior of the final product will be different.

Another new contribution is the fact that in the polyol method only polyvinylpyrrolidone (PVP) has been used as capping agent. It is demonstrated herein that other compounds, such as polydiallyldimethyl ammonium chloride (PDDAM), can be used as capping agents without modifying significantly the system characteristics. The observation that other capping agents may be used, opens the possibility to expand the type of compounds that are used as capping agents, so more specific properties may arise.

Synthesis of nanowires. The setup used for this study included a three neck flask with condenser system heated in oil bath. 10 ml of ethyleneglyco (EG) or glycerin (G) with 5 ml of an EG or G solution of 0.375 M PVP or PDDAM were refluxed at 160° C. for 2 h, then 5 ml of an EG or G solution of 0.25 M silver nitrate were added drop wise in not less than five minutes. The color of the solution changes significantly in the study. When the first drops of silver nitrate are added, the mixture turns yellow immediately. With the addition continuing, the solution becomes opaque gradually. After all the silver nitrate solution is added, the solution turned turbid with a grey color in about 15 min, indicating the appearance of Ag nanowires; the reaction continued at 160° C. for 30 min. When the reaction has finished, centrifugation is needed to remove nanoparticles and other impurities from the nanowires. The grey precipitate remained and needed no further purification.

Initial characterization of nanowires. The samples were characterized by Scanning Electron Microscope (SEM), Transmission Electron Microscope (TEM) and X-Ray Diffraction (XRD). The product from the reaction is always a combination of nanoparticles and nanowires. An interesting difference between PVP and PDDAM is that the nanoparticles generated by the use of the later are very sensitive to the irradiation produced by the electron beam. When PVP is used, the nanoparticles have polyhedral and cubic shapes while in the case of PDDAM only cubes are formed. In the case of the nanowires, they have a five-fold symmetry. They have an average diameter of around 70 nm and lengths up to several microns. It is important to mention that the aspect ratio and the production rate of the nanowires can be controlled to some extent modifying the reactions conditions. X-Ray diffraction of two typical samples showed that when PVP is used with either EG or G, only pure silver compounds are synthesized (nanoparticles and nanowires). In the case of PDDAM (FIG. 2b) two different phases are present: silver and silver chloride. This is consistent with the fact that the nanoparticles formed using PDDAM as capping agent are very sensitive to the electron beam, while the nanowires remain intact. Further TEM analysis may be conducted for the PDDAM systems the nanoparticles are mainly AgCl and the nanowires are only Ag.

TEM analysis demonstrated that for all cases (EG or G and PVP or PDDAM) the obtained nanowires have the same structure, being single crystalline and having several defects like twin boundaries, dislocations and stacking faults. EDS analysis on different samples showed that they are composed only of Ag and have a very thin amorphous layer of capping agent (PVP or PDDAM). Further analysis to fully understand the growth mechanism and the role that the defects play on it is in progress. However, it is expected that these type of nanowires will have mechanical properties that strongly deviate from the bulk material.

These results confirm that the polyol method is a very reliable route to synthesize silver nanowires. It is also demonstrated herein that several modifications to the reported method can be 10 made and silver nanowires can still be synthesized. In fact, the present invention demonstrates the use of new solvents and capping agents heretofore not used with nanoparticles.

Some of the main conclusions may be summarized as follows:
a) Both, PVP and ethylene glycol can be replaced by PDDAM and glycerin and silver nanowires can still be synthesized.
b) When PVP is used as a capping agent, the addition rate of silver nitrate is very critical. Only addition rates lower than 0.5 mL of AgNO3/min can produce high aspect ratio nanowires.
c) In PDDAM+glycerin system, the molar ratio of capping agent could be reduced from 3:2 (PVP:Ag) to 1:4 (PDDAM:Ag). However, because of the chlorine present in PDDAM, AgCl nanoparticles are also produced.
d) At the same reaction conditions and a molar ratio of PVP:Ag of 6:1, glycerin produces more nanowires than ethylene glycol.
e) When glycerin is used instead of ethylene glycol, PDDAM produces more nanowires than PVP.
f) In the case of PVP, the nanowires can be separated from the nanoparticles by centrifugation using acetone to dilute the sample, while for PDDAM is not possible to separate the nanowires from the nanoparticles by this procedure.

As opposed to the most extensively used synthesis method (known as the polyol method) which uses ethylene glycol as the solvent and reducing agent, the glycerin method disclosed herein eliminates the problem of finding an agent which will facilitate the contact of the silver nanoparticles with the body. The reason is because glycerin is a lot friendlier with the human body than ethylene glycol. That is why glycerin, in contrast to ethylene glycol, plays an important role in a vast range of body care products. Use of well-known and characterized compounds that are biocompatible and non-allergenic is especially important because of the possibility to generate products to prevent the infection against, e.g., HIV.

In operation, the present invention may be further characterized as follows. In recent years, inorganic nanostructures have attracted growing interest due to their potential applications in catalysis¹, biological sensors², and nanoelectronics³ among others. As these materials have at least one of their dimensions between 1 nm and 100 nm, interesting properties arise due to phenomena such as quantum confinement and high surface-to-volume ratio⁴.

In the case of noble-metal nanomaterials, the physicochemical properties are highly influenced by shape and size^{1,5}. For example, it is well known that the optical absorption spectra of metal nanoparticles are dominated by surface plasmon resonances (SPR)⁶, being the case of gold and silver unique. Both have the proper density of free electrons for their nanoparticles to possess SPR peaks in the visible region of the electromagnetic spectrum⁷, which in addition to their large effective scattering cross section, makes them ideal candidates for molecular labeling⁸. Recently, the absorption spectra of individual silver nanoparticles was correlated with their size and shape determined by transmission electron microscopy (TEM)⁹. The results indicate that spherical and roughly spherical nanoparticles absorb in the blue region of the spectrum, while decahedral nanoparticles and particles with triangular cross sections absorb in the green and red part of the spectrum, respectively. For each different morphology, the SPR peak wavelength increases with size. Thus, an exquisite control of size, composition and morphology is highly desirable. Additionally, we recently found that the size of silver nanoparticles is important in other applications by demonstrating that silver nanoparticles undergo a size-dependent interaction with HIV-1, with nanoparticles exclusively in the range of 1-10 nm attaching to the virus¹⁰. Due to this interaction, silver nanoparticles inhibit the virus from binding to host cells.

Noble-metal nanocrystals have been synthesized using a variety of methods, being the solution phase techniques probably the most employed ones¹. In solution phase synthesis of gold and silver nanoparticles the control of shape is a challenging task, since the structure of the original seeds plays a crucial role in the morphology of the final product¹¹. Multiple TEM studies on small (<5 nm) metal particles¹²⁻¹⁶ have demonstrated that small excitations, even at room temperature, may be sufficient to induce structural fluctuations, and that the rates of such fluctuations increase with the decreasing size of the nanocrystal¹³. These morphological fluctuations have also been validated by other techniques such as infrared spectroscopy¹⁷. Based on these observations and the results of theoretical calculations, it is known that the total potential surface energy for the possible nanoparticle morphologies consists of several minima, and that the barriers between them are low enough (˜kT), so that thermal fluctuations provide sufficient energy to produce changes between the different morphologies. At these small sizes, the five fold symmetry twinned structures such as the icosahedron and the decahedron tend to be slightly more stable, while cuboctahedral nanoparticles become more stable at larger sizes¹². It is also known that when the size of the nanocrystals increases the structural fluctuations cease, thus fixing the nanoparticle morphology either as single-crystalline or multi-twinned¹¹. On the basis of this model, one can easily understand why in a typical sample of nanoparticles produced by most of the solution phase techniques, a statistical distribution of shapes will be observed. However, significant progress has been achieved in controlling the shape and size of the metal nanostructures, and many applications have been proposed based on their morphology¹⁸⁻²¹.

Among solution phase techniques, the polyol method is one of the most employed routes for the synthesis of metal nanostructures. In this method, a metal precursor is dissolved in a liquid polyol, e.g. ethylene glycol, in the presence of capping agent molecules such as poly-vinyl pyrrolidone (PVP). By controlling parameters such as the molar ratio between metal precursor and capping agent, order of addition, temperature and reaction time, a reasonable control of size and morphology can be achieved²². In the case of silver, Xia and coworkers demonstrated that by carefully adjusting some of these variables and particularly the molar ratio between silver nitrate and PVP, a range of controlled morphologies such as nanocubes and nanowires can be produced¹¹. They found that if single-crystalline seeds were primarily produced, the final product is composed of monodispersed cubes, tetrahedrons and octahedrons, while if multi-twinned particles (MTPs) with decahedral shape were primarily formed; the final product is mainly composed of nanorods and nanowires with a remarkable multi-twinned structure with pentagonal cross sections. The fact that 1-D structures of silver can be reliably produced in high yields is quite significant, since the synthesis of anisotropic fcc materials is not as simple as for non-cubic crystal structures.

Additionally, Gao, et al., found that when the molar ratio between PVP and AgNO₃ is six to one, only a monolayer of PVP is adsorbed into the surface of the wires²³. Therefore, it may be expect that when the nanowires are exposed to air, the polymer membrane will be permeable enough to atmospheric gases and water vapor. Thus, the stability of the nanowires exposed to air against atmospheric corrosion needs to be explored. Furthermore, the higher reactivity of metal nanomaterials compared to their bulk state is also commonly known, so phenomena such as corrosion might be enhanced.

Atmospheric corrosion in metals is a frequent phenomenon. Unlike other metals, atmospheric corrosion of silver, also known as tarnishing, occurs towards sulfidation and not the formation of silver oxide²⁴. The atmospheric sulfidation of silver has been extensively investigated²⁴⁻²⁸. It has been demonstrated that silver sulfidizes upon exposure to several gaseous sulfur-containing compounds, being hydrogen sulfide (H₂S) and carbonyl sulfide (OCS) the most important silver corrodents²⁵. Surprisingly, the study of atmospheric corrosion in the type of silver nanostructures described so far is an area that has been largely unexplored. To our knowledge, there is no information about the stability of these nanostructures after they are extracted from the synthesis solution and exposed to air.

The present inventors tested the stability of PVP-coated silver nanowires synthesized by the polyol method when they are extracted from solution and exposed to air at ambient conditions. The stability of such silver nanostructures is demonstrated when a thin layer of silver sulfide nanocrystals is formed on the surface of the nanowires. Along with these findings regarding the stability of the PVP-coated silver nanostructures, a new method for the production of core-shell silver-silver sulfide nanoparticles and nanowires is presented.

Metal nanostructures such as nanoparticles and nanowires have been proposed as building blocks for several applications in nanofabrication and nanoelectronics. However, even when atmospheric corrosion is common in metals, there is a lack of information about the stability of those nanostructures against such phenomenon. Therefore, atmospheric corrosion of silver nanowires and nanoparticles synthesized by the polyol method using poly-vinyl pyrrolidone (PVP) as capping agent by different techniques, including transmission electron microscopy (TEM) and x-ray photoelectron spectroscopy (XPS) was determined. After synthesis and purification, the silver nanostructures were deposited on different substrates and exposed to laboratory air at ambient conditions. The structural changes in the samples were monitored by TEM as a function of time for a period of time of twenty four weeks. These results demonstrate that these silver nanostructures are susceptible to atmospheric corrosion and that, in most cases, a thin layer of silver sulfide nanocrystals is formed on their surfaces. The enhanced reactivity of regions with defects and dislocations could explain the observation that the corrosion rate of the nanowires is higher than the corrosion rate of the nanoparticles, since it is well known that the structure of the nanowires synthesized by the polyol method is multi-twinned, while most of the nanoparticles that remained after synthesis are single-crystals. Additionally, part of the original sample of silver nanostructures was sulfidized using hydrogen sulfide (H₂S) as corrodent gas. After performing XPS studies of this sample, the presence of PVP on the surface of the sulfidized silver nanostructures was confirmed. This result agrees with the observation that in the atmospherically corroded samples, even when in some cases the original silver nanostructure was completely corroded, the silver sulfide nanocrystals remained together adopting the shape of silver nanostructure. Finally, these results indicate that the corrosion at the nanoscale seems to be similar to that of the bulk silver.

Materials. Silver nitrate and poly (N-vinyl-2-pyrrolidone) (PVP-K30, MW=40,000) were purchased from Sigma Aldrich and ethylene glycol was purchased from Fischer Chemicals. Deionized water was prepared with a Milli-Q water purification system. All the materials were used without any further treatment.

Synthesis of silver nanoparticles and nanowires. Silver nanoparticles and nanowires were synthesized by reducing silver nitrate (AgNO₃) in ethylene glycol in the presence of poly-vinyl pyrrolidone (PVP). In a typical synthesis, 5 mL of pure ethylene glycol and 5 mL of a 0.36 M solution of PVP in ethylene glycol were refluxed in a three-necked flask at 160° C. with vigorous stirring for about 60 minutes. After that, 2.5 mL of a 0.12 M solution of AgNO₃ in ethylene glycol was injected drop-wise into the reaction flask. After 60 minutes the reaction was stopped, allowing the product to cool to room temperature. A mixture of silver nanoparticles and nanowires was obtained. The reaction product was diluted in deionized water (5× in volume). The silver nanowires were purified by centrifugation.

Stability of silver nanoparticles and nanowires. The stability of the synthesized nanowires against atmospheric corrosion was evaluated by electron microscopy. Samples for scanning and transmission electron microscopy were prepared just after the product was synthesized. All the samples were exposed to lab air at ambient conditions (23±2° C. and a relative humidity of 70±4%). Electron microscopy analyses were periodically conducted during a time interval of six months.

Sulfidation of silver nanoparticles and nanowires. The sulfidation experiments were carried out in a controlled temperature tube-furnace. Samples of the purified reaction mixture were placed inside a quartz tube on different substrates, i.e. TEM Cu grids, glass slides and (100) Si-wafers. Before use, the silicon and glass substrates were ultrasonically cleaned using ethanol and followed by a double de-ionized water treatment. Next, the samples inside the quartz tube were slowly heated up to 100° C. in a continuous nitrogen (N₂) flow of 10 sccm. Once the target temperature was reached, a 10 sccm continuous flow of hydrogen sulfide (H₂S) was allowed to run through the tube. After five hours, the H₂S flow was stopped and purge with N₂ for fifteen minutes. Finally, the tube was cooled to room temperature in atmospheric conditions.

Characterization of the samples. Scanning electron microscopy (SEM) was conducted using a Hitachi 4500F microscope operated at 15 kV. TEM analysis was performed in a JEOL 2010F microscope operating at 200 kV equipped with a Schottky-type field emission gun, an ultra-high resolution pole piece (Cs=0.5 mm), an energy dispersive x-ray spectrometer (EDS) unit, and a scanning transmission (STEM) unit with high angle annular dark field (HAADF) detector. Samples for TEM were prepared by depositing a drop of the original suspension on a lacey carbon coated Cu grid and allowed to evaporate. Crystal structure identification by X-ray diffraction (XRD) was carried out in a Phillips automated vertical scanning powder diffractometer. The spectrum was obtained from 10 to 70 2θ degrees. X-ray photoelectron spectroscopy (XPS) was conducted in a PHI 5700 system equipped with dual Mg X-ray source and monochromated Al X-ray source with depth profile and angle resolved capabilities. The spectra were fitted using Gaussian curves. Samples for SEM, XRD and XPS were prepared by covering a substrate (Si for SEM and XPS, and amorphous glass for XRD) with the original suspension and letting the water to evaporate. In the case of the sulfidized products, the samples were analyzed directly from the substrates (TEM Cu grids, glass slides and Si-wafers) used in the reaction.

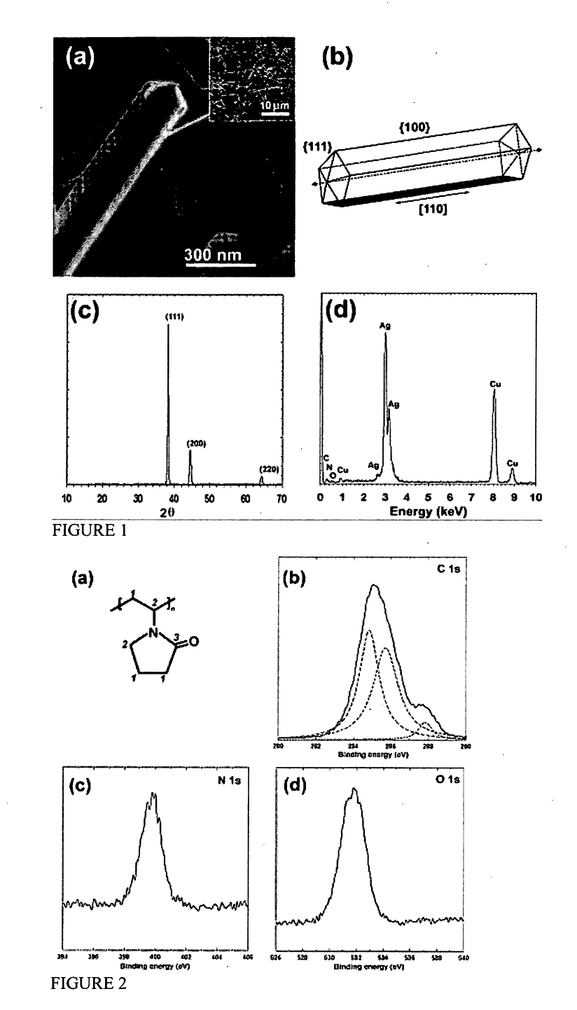

Characterization of the as-synthesized silver nanowires. As shown in the inset of Figure la, the product after purification was mainly composed by nanowires. Compositional analysis performed under the TEM by acquiring EDS spectra of several nanowires, revealed that the body is only composed of silver, while the presence of PVP in the surface of the nanowires is responsible for the appearance of the characteristic carbon, nitrogen and oxygen peaks (FIG. 1d). X-ray diffraction of the synthesized nanowires (FIG. 1c) showed just a single crystalline phase that could be indexed as fcc silver (JCPDS File 08-0720). As a direct result of the 1-D structure of the nanowires, the ratio between the relative intensities of the {111} and {100} planes is higher than the expected for bulk silver (˜4.6 vs. ˜2.2), indicating the relative abundance of {111 } planes.

As mentioned before, the structure of these nanowires is quite notable. Their tips resemble a decahedral nanoparticle and exhibit a pentagonal cross-section all across their long axis (FIG. 1b). Based on that, it has been proposed that these nanowires evolve from a multi-twinned decahedral nanoparticle growing in the [110] direction with the capping agent guiding the structure by stabilizing more effectively the newly formed {100 } facets than the {111 }²⁹. In a different report, Sun et. al. found that small gold nanoparticles functionalized with dithiol linkages preferentially attached to the ends of the nanowires (i.e. the {111 } facets), which demonstrates again that PVP interacts more strongly with the {100} facets in the main body of the wire³⁰.

However, the role of the capping agent is not limited to the growth mechanism. Once the products are formed, it provides stability by protecting the surface of the nanocrystals. Additionally, the capping agent can modify the interactions of the metal nanostructure with external systems. In fact, the physicochemical properties of nanostructures are strongly dependent upon their interactions with capping agent molecules³. Therefore, it is important to understand the interaction between the capping agent and the surface of the nanowires.

In order to have a better understanding about the adsorption of PVP on the surface of the nanowires, x-ray photoelectron spectroscopic (XPS) studies were conducted. The results for pure PVP and PVP-coated silver nanowires are presented in Table 1.

TABLE 1
Binding energy values of pure PVP
and PVP-coated silver nanowires

| | Binding energy (eV) | | | | |
|---|---|---|---|---|---|
| Sample | C 1s* | N 1s | O 1s | Ag 3d_{5/2} | Ag 3d_{3/2} |
| Pure PVP | 284.8 (1) | 399.9 | 531.8 | — | — |
| | 285.7 (2) | | | | |
| | 287.8 (3) | | | | |
| PVP-coated | 284.8 (1) | 399.8 | 531.9 | 367.8 | 373.8 |
| silver | 285.6 (2) | | 532.9 | | |
| nanowires | 287.8 (3) | | | | |

*The number in parenthesis corresponds to the different carbon species in the PVP repeating unit according to FIG. 2a.

All the binding energies are referenced to C 1s (284.5 eV). PVP is a linear polymer that has a polyvinyl skeleton with polar groups forming a pyrrolidone ring. Based on that, three carbon species with different chemical environments can be identified. For the purposes of our analysis, we labeled them as 1, 2 and 3 (FIG. 2a). In the case of pure PVP, the spectrum obtained for C 1s can be deconvoluted into three peaks with binding energies of 284.8, 285.7 and 287.8 eV, while the N 1s and O 1s spectra exhibited single peaks at 399.9 and 531.8 eV, respectively (FIG. 2). The fitted C 1s peaks can be explained in terms of the electronegativity of the substituents of the different carbon atoms that compose the PVP repeating unit. In general, as the electronegativity of the substituent is higher the more it withdraws electron density from the carbon atom, causing an increase in the binding energy of the C 1s electrons. The carbon atom numbered as three is bonded to oxygen, which is the element with the highest electronegativity in the pyrrolidone ring. Therefore, the peak at 287.8 eV can be attributed to the C═O bond, while the 285.7 and 284.8 eV peaks are result of the C—N and C—C bonds, respectively.

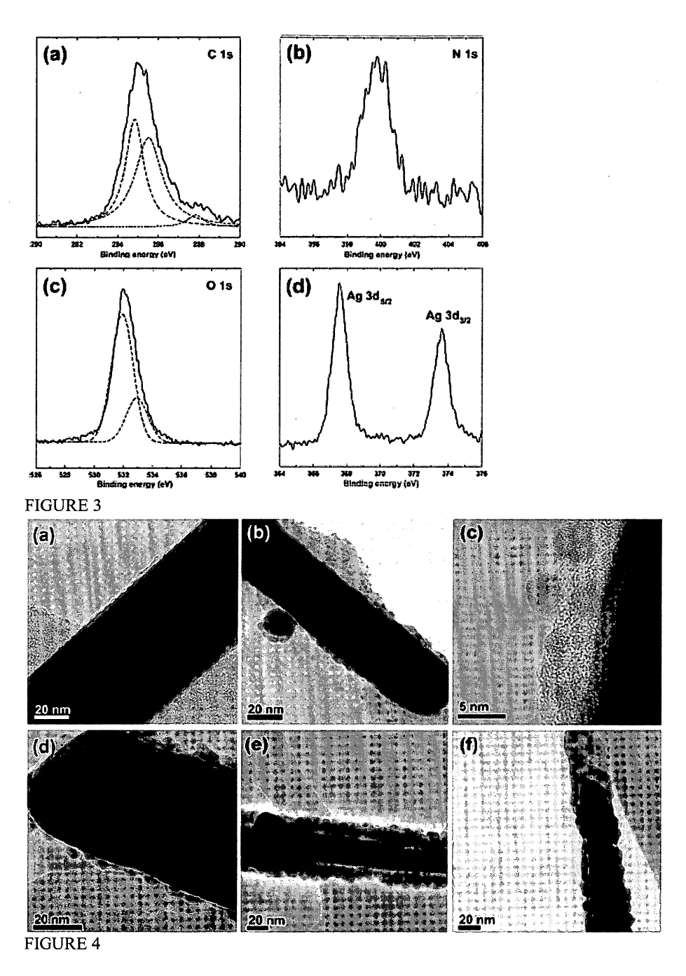

By analyzing the XPS spectra of the PVP-coated silver nanowires, no significant differences with respect of pure PVP can be observed in the C 1s and N is peaks (FIG. 3). However, the peak of O 1s can be deconvoluted in two peaks with binding energies of 531.9 and 532.9 eV. In addition, the observed peaks for silver resulting from the Ag 3d_{5/2} and Ag 3d_{3/2} electrons have binding energies of 367.8 and 373.8 eV, respectively. The 0 is peak at 531.9 eV is similar to the one observed for pure PVP, while the peak with binding energy of 532.6 eV can be attributed to the interaction between the oxygen in the carboxyl group of the PVP chain and the surface of the silver nanowires. It is probable that the interaction with the surface of the nanowire decreases the electron density of the oxygen atom in the carboxyl group, producing the appearance of the peak at 532.6 eV. By analyzing the XPS spectra of PVP-coated silver nanoparticles, Huang et. al. proposed that the adsorption of the oxygen atom in the carboxyl group on the silver nanoparticle surface will induce an image dipole on the particle surface, so the observed binding energies for the 0 is and the Ag 3d_{5/2} electrons are dominated by the electrostatic interaction between the final silver ions with their environment; producing an upper shift in the case of the O 1s binding energy, and lowering the binding energy of the Ag 3d_{5/2} electrons with respect of that for bulk silver³¹. Furthermore, the binding energies for the Ag 3d_{5/2} and the Ag 3d_{3/2} electrons are smaller than the binding energies of metal silver (368.2 eV for Ag 3d_{5/2}, and 374.2 eV for Ag 3d_{3/2}) but higher than the binding energies of silver (1) oxide (367.5 eV for Ag 3d_{3/2}, and 373.5 eV for Ag 3d_{3/2})³². This is also a clear indication of the strong interaction between the oxygen atom of the carboxyl (C═O) groups in the PVP chain and the silver surface of the nanowires.

Stability of the silver nanowires. The stability of the synthesized silver nanowires against atmospheric corrosion after exposure to air at ambient conditions was evaluated by electron microscopy. Electron microscopy and their related techniques are suitable for this type of analysis mainly because they provide the ability to study in great detail the structural properties and compositional characteristics of many different materials. As mentioned in the experimental section, samples for TEM analysis were prepared just after the product was synthesized and analyzed periodically during an interval of six months.

FIG. 4 includes electron microscopy images of the same TEM sample of silver nanowires analyzed at different times. FIG. 4a shows the condition of the silver nanowires just after synthesis. As can be observed in the image, the surface of the nanowire is smooth and the presence of a twin boundary in the middle of the nanowire is clear. This twin boundary results from the five-fold symmetry of these type of nanowires. FIG. 4b presents an image of the same sample after three weeks of exposure to ambient air. The surface of the nanowire is rougher than the observed in panel a, and nanoparticles start to appear on the surface and the surroundings of the wires. A higher magnification of the nanoparticles on the surface of the nanowires is shown in FIG. 4c. The crystalline nature of such nanoparticles is evident from the image. In panels d, e and f the same sample after exposure to air for four, five and twenty four weeks respectively, can be observed. Both panels e and f present similar structural changes, where a shell of crystallites is formed around the original silver nanowires. The diameter of the core-shell structure matches the diameter of the original nanowire, i.e. the diameter of the silver nanowire core is decreased.

The same analysis was conducted by scanning electron microscopy (SEM) and the results are presented in FIG. 5. In FIGS. 5a and 5b images of the sample after four weeks are shown. It can be observed that small protuberances, that resemble the ones presented in FIG. 4d, start to appear. In some cases, after twenty four weeks (FIG. 5c and 5d) the structure of the nanowires can not be clearly discerned. Several nanowires seem to have coalesced and the surfaces are more irregular and rough. Also, some nanowires present fractures along their length. Additionally, at the ends of several nanowires the presence of larger bumps is observed. Clearly, the as-synthesized nanowires are not stable and a notable degradation is observed.

It is well known that when a highly energetic electron beam passes through a sample, electrons lose energy predominantly through electronic and nuclear interactions with the specimen. These interactions may damage the structure of the sample. The damage caused by electronic interactions is known as radiolytic, while the damage produced by nuclear interactions is known as knock-on damage³³. Therefore, to properly study the observed structural changes, it was important to confirm that they were not direct consequence of the irradiation damage produced by the periodical observation in the TEM. For the case of the silver nanowires, the irradiation damage produced in the metal core by the TEM electron beam at 200 kV should be negligible. However, the silver nanostructures analyzed herein were capped by an organic polymer that can be damaged by the exposure to periodical electron irradiation.

FIG. 6 shows TEM images of samples that were not exposed to periodical electron irradiation. The TEM samples were prepared using product from the same solution batch of the silver nanowires presented in previous figures. FIGS. 6a and 6b present images of two different samples observed after six and twenty four weeks, respectively. Both images show that the degradation results are congruent with the ones presented in the previous discussion, and that a thin layer of crystallites is formed on the surface of the nanowires. Thus, the observed structural changes are not generated by the periodical irradiation of the electron beam. The fact that no significant irradiation damage was observed can be explained by the fact that for a material to experience knock-on displacements a specific energy threshold needs to be surpassed. Below this energy, the electron beam just enhances atom vibrations in the sample and the provided energy is dissipated as phonons. For most metals, the threshold energy is 20-30 eV and, unless long exposures and or high current densities are achieved, knock-on damage does not occur for accelerating voltages less than 300 kV³³. In the case of radiolytic damage, the ionization effects decrease significantly as the acceleration voltage of the electron beam increases up to 100 kV and remain low at higher voltages³³.

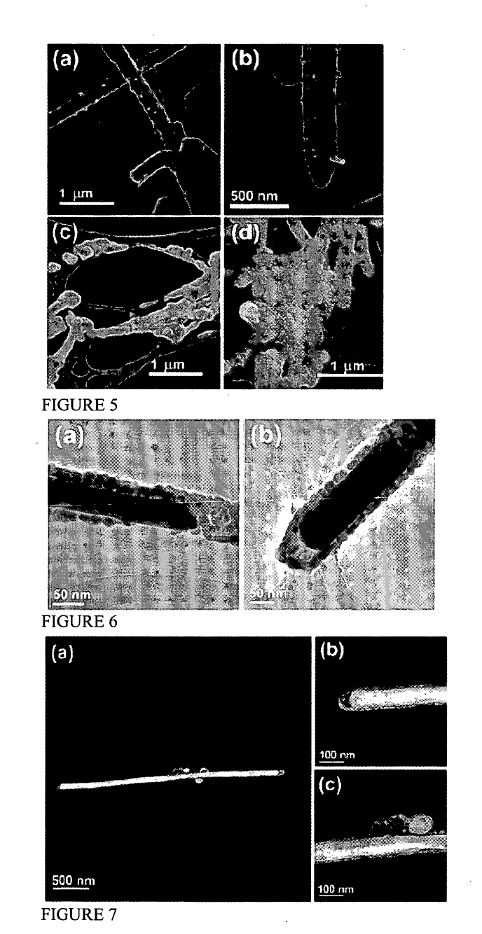

FIG. 7 presents high angle annular dark field (HAADF) images of the nanowire that appeared in FIG. 6b. It is clear that the shell of nanocrystals covers all the surface of the nanowire, having a regular thickness of ˜15 nm across the length of the wire. Similarly to some of the previous images, at the tips of the nanowire a low-density region appears between the shell and the core. The bright contrast in the exterior of the shell might be due to the increased thickness because of the cylindrical shape of the shell, while in the low-density region the electron beam could be just traveling across two ˜15 nm thick cylindrical walls of the shell, finding a hollow center. Interestingly, the formation of this shell of crystallites is also noticeable in two nanoparticles that remained attached to the original nanowire (Panels a and c). In the case of the nanoparticles, the thickness of the shell is also of ˜15 nm. Three regions without the brighter core are also distinguishable. The observed contrast suggest that these are hollow structures only composed by a shell of nanocrystals. It is important to note that all these observations suggest that the PVP coating is still there, promoting that all the nanocrystals that composed the shell remain together adopting the shape of the original silver nanostructure.

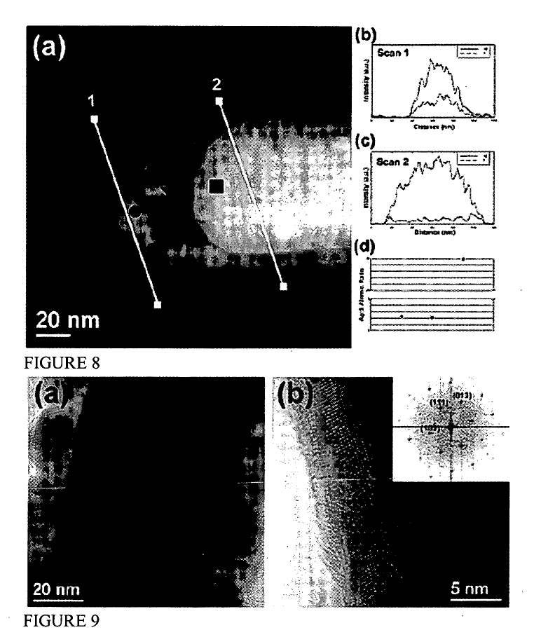

Compositional analysis of three different regions in one of the tips of this nanowire demonstrated the presence of considerable amounts of sulfur (FIG. 8). Energy dispersive x-ray spectroscopy line scans were acquired for two different regions. In this case, the intensity of the profile is proportional to the concentration of the element that is being analyzed. The first line scan is presented in FIGS. 8a and 8b, and corresponds exclusively to the shell of nanocrystals. As observed in FIG. 8b, the intensity profile of sulfur seems to be half of the profile obtained for silver, suggesting that the shell is composed by silver sulfide (Ag₂S) crystallites. In the case of the second line scan (FIGS. 8a and 8c), the proportion of silver compared to sulfur is higher. This suggests that the bright core of the nanowire is still pure silver, while just a thin layer of silver sulfide nanocrystals was formed on its surface. Interestingly, the ratio between the silver and the sulfur intensity profiles at both ends of the scan is again close to 2:1. This is congruent with the fact that, at both ends, the line scan corresponds just to the shell. To determine more precisely the atomic ratio between silver and sulfur in different regions of the wire, punctual compositional analyses were acquired operating the electron microscope in EDS mode. The results are presented in FIGS. 8a and 8d. In the case of the shell, the atomic ratio between silver and sulfur is close the stoichiometric of silver sulfide. The measured ratio of the outer part in the shell was of 2.25 while in the low-density region of the shell was of 2.09. For the case of the bright core of the nanowire, the atomic ratio between silver and sulfur was of 29.90, confirming that the core is formed by pure silver surrounded by a shell of silver sulfide nanoparticles.

Analysis of the Fast Fourier Transform obtained from a high resolution TEM image of the crystallites surrounding the silver nanowires confirmed also the presence of silver sulfide. The analysis of the spots presented in the FFT of FIG. 9b indicated the presence of a single crystalline phase. As presented in Table 2, the interplanar distances and angles between planes measured from the FFT are in good agreement with monoclinic acanthite silver sulfide, which is the most stable polymorph of silver sulfide at room temperature. This agrees with the fact that in previous studies the silver sulfide resulting from the corrosion of silver is primarily crystalline rather than amorphous²⁸.

TABLE 2
Interplanar distances and angles between planes
for reflections of the FFT presented in FIG. 9b.

| | Measured values | Reported values* |
|---|---|---|
| d₍₀₁₃₎ (Å) | 2.42 | 2.42 |
| d₍₁₁₁₎ (Å) | 3.05 | 3.08 |
| d_{(10\overline{2})} (Å) | 3.11 | 3.11 |
| (013) ∠ (111) | 49.1° | 50.0° |
| (111) ∠ (10\overline{2}) | 79.2° | 79.4° |
| (\overline{1}02) ∠ (013) | 51.7° | 50.6° |

*According to monoclinic acanthite silver sulfide (JCPDS File 89-3840) with cell constants a = 4.23 Å, b = 6.91 Å, c = 7.87 Å and β = 99.58°.

It is known that the principal product from the atmospheric corrosion of silver is silver sulfide, and that the corrosion layers do not include carbonates, sulfates or nitrates²⁸. The silver corrosion process is primarily influenced by the type and the amount of reduced-sulfur gases such as H₂S, OCS, SO₂ and CS₂ present in the atmosphere, as well as the amount of water on the silver surface²⁴⁻²⁸. It has been reported that among the reduced sulfur-containing gases, H₂S and OCS are the principal corrodents of silver, since the sulfidation rates of those gases are about one order of magnitude higher than the rates of SO₂ and CS₂²⁵. Even though the typical concentrations of these reduced-sulfur gases in the atmosphere are low, they are sufficient to initiate the corrosion process²⁸.

In the case of the reaction between silver and hydrogen sulfide, the sulfidation mechanism needs to be completely understood²⁶. It is believed that the general reaction between them occurs according to reaction (1), and that the water present on the silver surface provides the proper medium for the gas to be dissolved for the subsequent reaction with silver. Indeed, extensive research has demonstrated that the silver corrosion increases with increasing relative humidity^{24-26,28}.
2 Ag+H₂S→Ag₂S+H₂ (1)

Also, the presence of other gases, such as O₂ and NO₂ can enhance the sulfidation process according to the following reactions²⁷:
2 Ag+H₂S+/½O₂→Ag₂S+H₂O (2)
2 Ag+H₂S+2 NO₂→Ag₂S+2 HNO₂ (3)

When no sources of H₂S are available, OCS is the principal corrodent of silver in atmospheric conditions²⁵. As shown in reaction (4), in the presence of water, this gas rapidly decomposes to form hydrogen sulfide. The corrosion produced by OCS is important since it is the most abundant sulfur species in the atmosphere.
OCS+H₂O→H₂S+CO₂ (4)

Another interesting point, as demonstrated by these results, is that when silver is corroded, it does not tend to form uniform films. Silver sulfide is rather grown as a rough, discontinuous series of clumps²⁸. Bennet, et al., found that the silver sulfide clumps formed on the surface of evaporated silver films coalesce slowly into a continuous film²⁴. They reported that in atmospheric air with concentrations of H₂S as low as 0.2 parts per billion, a 1.5-3 nm-thick non-uniform tarnish film is formed after one week, and a 6 nm-thick or more after one month. However, the corrosion rate is highly sensitive to other variables such as relative humidity and temperature, so different rates to the ones presented by Bennet can be achieved at different conditions.

In FIG. 10, images of different regions of the sample shown in FIG. 6b are presented. From the images; it is clear that not only the nanowires are being corroded but also the nanoparticles. Interestingly, not all the nanowires are being corroded in the same manner. As presented in panels e and f, the corrosion of some nanowires is not homogeneous. In these cases certain regions of the nanowires are corroded at higher rates than others. Without creating any limitations, two reasons can contribute to this phenomenon. The first one can be related to a lower localized PVP surface coverage that facilitates the corrosion process, and a second reason can be the existence of regions with higher proportion of defects along the nanowires. As regions with defects are more reactive, the corrosion process might be enhanced by the presence of such defects.

Indeed, the enhanced reactivity of regions with defects can also explain why the corrosion rate of the nanowires seems to be higher than the nanoparticles. As mentioned earlier in the manuscript, it was found that single-crystalline seeds primarily produced cubic, tetrahedral and octahedral nanoparticles, while multi-twinned seed particles (MTPs) with decahedral shape induce the formation of nanorods and nanowires with a multi-twinned structure with pentagonal cross sections. The basic structure of the decahedral seed particle can be described as the junction of five tetrahedron single crystals with twin-related adjoining faces³⁴⁻³⁸. The theoretical angle between two (111) planes is ˜70.5°, so by joining five tetrahedra a gap of ˜7.5° is generated. Thus, the space is not filled and some form of internal strain is necessary, giving place to dislocations and other structural defects^{38,39}. These defects are also observed in transmission electron microscope (TEM) cross-section images of the aforementioned nanowires^{29,40}.The presence of these defects across the length of the nanowires enhances their corrosion compared to the one observed for the single-crystalline nanoparticles, as the case of the nanocube presented in FIG. 10d. In fact, it has been demonstrated that twinned particles are expected to exhibit a stronger reactivity and susceptibility towards etching than single crystalline particles⁴¹. In the case of silver tarnishing, it is known that the presence of facets or steps in the silver crystal enhances the sulfidation rate^{42,43}. Nucleation and growth of silver sulfide occur more rapidly along defects and dislocations than on smooth defect-free surfaces⁴⁴.

Another important observation arises from the fact that in other cases, even when the nanowires have been completely corroded as in the case of panel b, the silver sulfide nanocrystals remained aligned in a wire-like form. Again, this implies that the PVP layer is still absorbed into the surface of the formed nanocrystals. However, the specific interaction between the atmospherically corroded products and PVP is yet to be determined.

On the other hand, nanowires that remained in the original solution for weeks were also evaluated by electron microscopy (images not shown here). After twenty four weeks, no corrosion was observed. Compositional analysis demonstrated that they are only composed by silver capped by PVP, and no sulfur was detected. This confirms that the corrosion occurs only when they are exposed to corroding sulfur sources, such as H₂S and OCS.

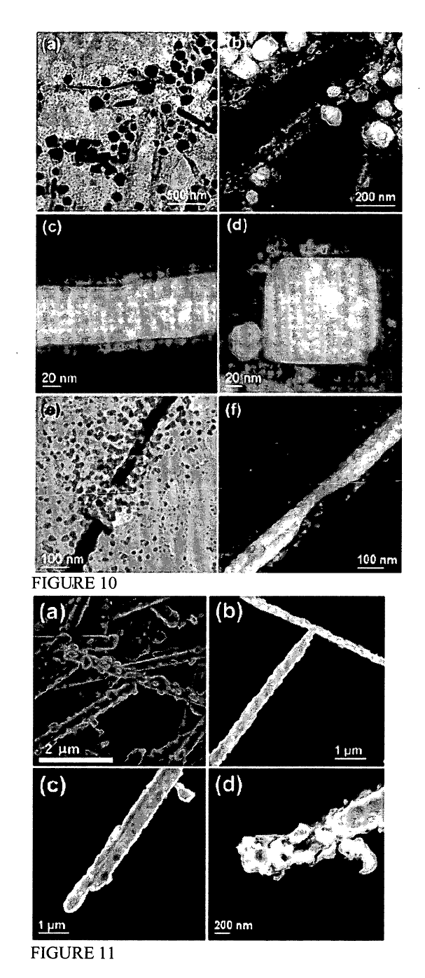

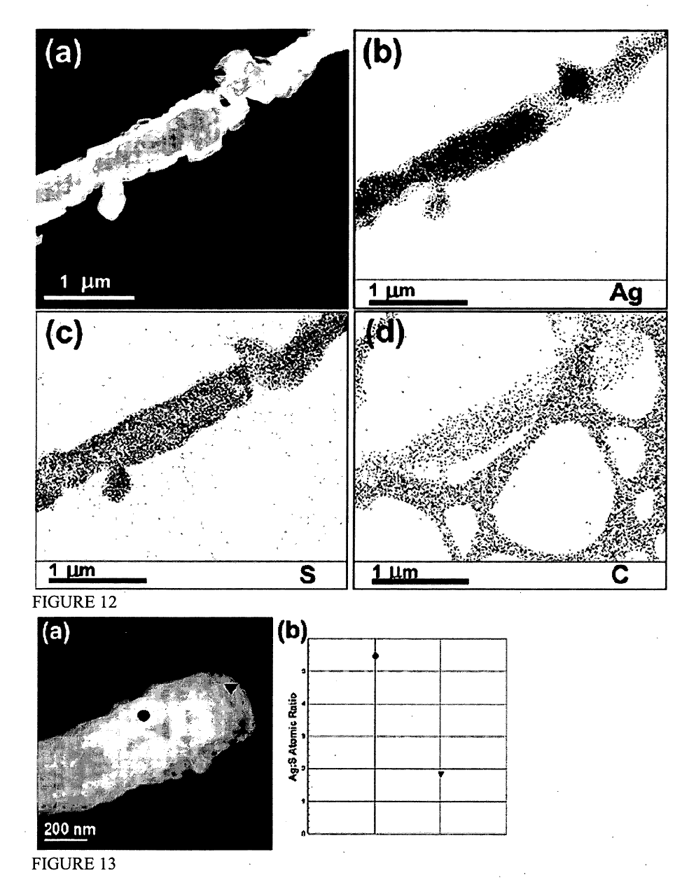

Sulfidation of silver nanostructures. As described in the experimental section, part of the original synthesized silver nanostructures were sulfidized in a nitrogen atmosphere using a 10 sccm flow of H₂S. The results are presented in FIG. 11. Larger crystalline bumps were produced on the surface of the nanowires. In some cases, as seen in FIG. 11d, the nanowire core was decomposed into several crystals presumably attached by the action of the PVP coating. Compositional EDS mapping was performed on one of these nanowires (FIG. 12). It is clear that the sulfur map matches the map obtained for silver, where the differences in intensities correspond to differences in relative concentration. Interestingly, the carbon map also correlates with the map of sulfur and silver. This is an indication that the PVP coating could be still present. As for the nanowires atmospherically corroded, punctual EDS analysis was conducted in the tip of one nanowire (FIG. 13). Two regions with markedly differences in contrast were analyzed and the atomic ratio between silver and sulfur was measured. The ratio between silver and sulfur in the brighter region was 5.47, while the ratio between them in the lighter region was 1.85. The fact that regions with ratios higher than the stoichiometric are detected indicates that those regions were not completely transformed into silver sulfide and that a metallic silver core is present.

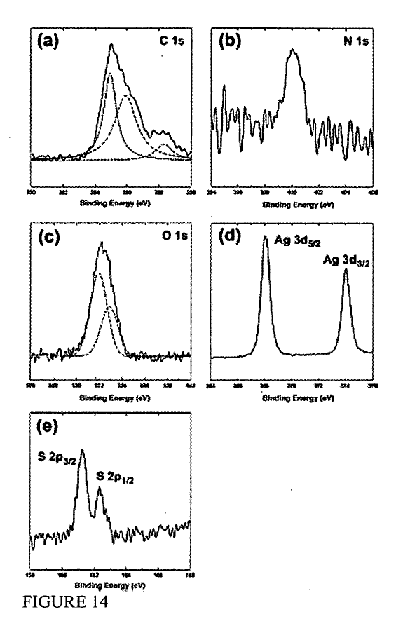

To confirm whether or not the PVP coating remains adsorbed on the surface of the nanostructures after they were synthetically corroded, XPS studies were performed (FIG. 14). The results are shown in Table 3.

TABLE 3
Binding energy values of the sulfidized silver nanowires
Binding energy (eV)

| C 1s* | N 1s | O 1s | Ag 3d_{5/2} | Ag 3d_{3/2} | S 2p_{3/2} | S 2p_{1/2} |
|---|---|---|---|---|---|---|
| 284.9 (1) | 400.0 | 531.9 | 368.0 | 374.0 | 161.2 | 162.3 |
| 285.9 (2) | | 532.8 | | | | |
| 288.3 (3) | | | | | | |

*The number in parenthesis corresponds to the different carbon species in the PVP repeating unit according to FIG. 2a.

The presence of C, N and O on the surface of the sulfidized nanostructures was confirmed. The values for the binding energies of the three elements were similar to the values obtained for the original silver nanowires, confirming the presence of PVP on the surface of the sulfidized nanowires. Again, the spectrum obtained for C 1s can be deconvoluted into three peaks with binding energies of 284.9, 285.9 and 288.3 eV, while the N 1s exhibited a single peak at 400.0 eV and the O 1s peak can be deconvoluted in two peaks with binding energies of 531.9 and 532.9 eV. This data suggest that PVP remains adsorbed on the surface of the sulfidized nanowires via the carboxyl group of the pyrrolidone ring. The observed peaks from the Ag 3d_{5/2} and Ag 3d_{3/2} electrons have binding energies of 368.0 and 374.0 eV, respectively, while the peaks corresponding to S 2p_{3/2} and S 2p_{1/2} have binding energies of 161.2 and 162.3 eV. These values are consistent with the reported data for silver sulfide³². In addition, the XPS data confirmed that no sulfites or sulfates were present. The binding energy of the O 1s peak also confirms that no silver (I) oxide was formed, since the expected binding energy for the 0 is peak in silver (1) oxide should be of 528.6 eV³², which is significantly lower than the value obtained for the sulfidized nanowires.

The silver nanostructures produced by the polyol method of the present invention using a six to one molar ratio between PVP and silver nitrate, are susceptible to atmospheric corrosion. In most cases, a thin shell of silver sulfide nanocrystals is formed on their surface. Multi-twinned nanowires are more vulnerable to corrosion compared to the single-crystalline nanoparticles due to their higher proportions of defects. Importantly, the fact that the presented silver nanostructures are being corroded at ambient conditions might limit their use in nanoelectronics and nanofabrication.

Additionally, it is well known that noble-metal nanostructures exhibit a phenomenon known as surface-enhanced Raman scattering (SERS) by which the scattering cross sections of adsorbed molecules are dramatically enhanced; thus, vibrational spectra for absorbates can be obtained^{45,46}. Because silver, gold and copper have appropriate values of the real and imaginary parts of the dielectric constants⁴⁷, SERS is usually conducted on roughened substrates of these metals. Alternatively, a powerful technique for the production of noble-metal nanoparticle arrays on different surfaces is the so called nanosphere lithography (NSL), where the resulting substrates are referred as metal film-over-nanosphere (MFON) surfaces⁴⁸. Based on the fact that silver nanostructures such as the ones presented here are susceptible to atmospheric corrosion, a careful evaluation of the effect that silver sulfide formation could have in SERS should be conducted. On the other hand, cleaning techniques such as hydrogen plasma reduction could help to reduce the formed sulfide to metallic silver. For example, hydrogen plasma was used to reduce silver sulfide on a daguerreotype surface by forming hydrogen sulfide, which was subsequently pumped away from the vacuum system⁴⁹. However, as for the case of silver sulfide growth rate, many variables such as the size and shape of the silver nanocrystals, the thickness of the sulfide layer and the time of exposure to the hydrogen plasma, among others, may affect the efficiency in the reduction of the sulfide. Thus, systematic studies should be performed on case-to-case basis.

It was further found that Ag—Ag₂S core-shell nanostructures can be produced, as described herein. This type of conductor-semiconductor nanostructures might be of interest for sensing purposes, since it has been shown that silver sulfide thin films with excess of silver can be used as photodetectors in the infrared region⁵⁰. Since the silver nanostructure acts as a template, the shape and size of the sulfidized nanostructures could be controlled.

Higher proportions of PVP could be used in the synthesis, therefore increasing the thickness of the polymer layer adsorbed on the surface of the nanostructures. The fact that a thicker layer of PVP is covering the silver surface of the nanostructures might reduce or prevent the corrosion of the metal core that forms the nanoparticle or the nanowire. Finally, silver nanomaterials such as nanoparticles and nanowires can be synthesized by many other colloidal techniques employing different capping agents. Independently of the synthesis method, after the silver nanostructures are exposed to sulfur-containing gas sources it is expected that they will be corroded. As noted previously, it is believed that water plays a fundamental role in the atmospheric corrosion of silver surfaces, so hydrophobic capping agents could improve the performance of these materials against corrosion. Thus, it is necessary to perform similar studies to the one presented here to properly evaluate the stability of the products generated by other synthesis techniques against atmospheric corrosion. In fact, this also applies to nanostructures of other metals that are subject to atmospheric corrosion such as iron and copper among others.

The Food and Drug Administration (FDA) has classified propylene glycol (glycerin) as an additive that is “generally recognized as safe” for use in food. It is used to absorb extra water and maintain moisture in certain medicines, cosmetics, or food products. It is a solvent for food colors and flavors. This advantage of glycerin being 100% friendly with the human body presents an advantage against other technologies. For the case of PVP, an extensive study of toxicological data in animals supports the biological inertness of PVP. The acute toxicity is extremely low and long-term administration has demonstrated no adverse effects. As a large molecule, it does not rapidly pass through most body membranes such as skin, or the gut wall. Studies have shown PVP to be essentially non-toxic by oral administration, inhalation and intravenous or other parenteral routes. PVP is not a primary irritant, skin fatiguing material or a sensitiser.

Nanoparticles produced by this technology do not present problems like instabilities or risky and expensive operation conditions that are found in physical methods for production of nanoparticles like sputtering or laser ablation. Nanoparticles produced by these methods need to be incorporated in a proper matrix before they can be used, while in the present invention incorporation into a matrix is not required.

The use of ethylene glycol, especially in body care products, is much more limited than the use of glycerin. Glycerin can be dissolved into water or alcohol, but not oils. On the other hand, many things will dissolve into glycerin easier than they do into water or alcohol. So the nanoparticles and related products are easily incorporated in a large variety of applications.

The fact that also nanowires can be produced opens a whole new set of opportunities because of the intrinsic properties that 1-D structures in the nanometric range have. Therefore, other applications may arise from these systems.

Vaginal Biocides: An agent (e.g., a chemical or antibiotic) that destroys microorganisms in the vagina. Research is being carried out to evaluate the use of rectal and vaginal biocides to inhibit the transmission of sexually transmitted diseases, including HIV. Like today's spermicides, a biocide could be produced in many forms, including; gels, creams, suppositories, films, or in the form of a sponge or a vaginal ring that slowly releases the active ingredient over time, that would give women the power to protect themselves from sexually transmitted diseases (STDs) and AIDS. Around the world women's health and lives are at risk every day because there are too few options in STD protection.

Disinfectant: A chemical which kills viruses and other microorganisms on a nonliving surface.

Biocide: a chemical substance, such as pesticides, which can be herbicides, insecticides, capable of killing different forms of organisms such as viruses used in fields such as agriculture, forestry, and mosquito control. Biocides can also be added to other materials (typically liquids) to protect them from biological infestation and growth.

Filters: to inactivate viral pathogens such as rotavirus in water or any liquid such as human milk from infected women of HIV-1.

Topical antiviral: eye drops or skin creams or gels.

Systemic antiviral: providing the nanoparticles systemically by delivering the nanoparticles intravenously, intramuscularly, subcutaneously, intradermally, transdermally, and the like.

The most important characteristic of the present invention is the use of silver nanoparticles as antivirals. The chemical and physical properties that bulk materials exhibit change drastically when the material is in the nanometer range. For this reason there is an increasing appeal in the development of nanomaterials, which can be used in physical, biological, biomedical and pharmaceutical applications.

Regarding the advantages in viral inhibition, it was found that silver nanoparticles are able to inhibit the HIV-1 virus in concentrations as low as 3 μg/mL. At this concentration, there is no toxicity on MT-2 cells (Human T-cell leukemia cells isolated from cord blood lymphocytes and cocultured with cells from patients with adult T-cell leukemia) and c-magi cells.

Finally, biocides containing silver nanoparticles would work in one of three ways: killing STD and AIDS viruses and bacteria, creating a barrier to block infection, or preventing the virus from replicating after infection has occurred. Ideally, biocides containing silver nanoparticles would be available either with or without spermicide in order to give women the option of becoming pregnant, while still protecting themselves from STDs.

The present invention relates to a method of inhibiting the transmission of Acquired Immunodeficiency Syndrome (AIDS) using silver nanoparticles.

The present invention provides an inexpensive, easily available and convenient method of inhibiting the transmission of the AIDS virus in humans as a result of sexual intercourse. The method relies upon the action of silver nanoparticles which results in a rapid killing action within minutes. These compounds are effective to reduce the infectivity of the AIDS virus and also kill the causative organisms of many other STD's after short exposure. The method of the invention is therefore useful to reduce the immediate risk of AIDS transmission. It also reduces future risk of AIDS transmission by eliminating STD causing organisms which increase the risk of AIDS.

The apparatus and method of the present invention is based on the finding that silver nanoparticles, are effective antiviral agents against retroviruses including the AIDS virus. Silver materials had previously been recognized as antibacterial agents useful in treating burns in man and animal. C. L. Fox, Jr., U.S. Pat. No. 3,761,590, relevant portions incorporated herein be reference. Silver in the form of AgSD has also been shown to be effective against certain viruses such as herpes simplex and herpes zoster and against the causative organisms of many STD's including Candida albicans, Treponema pallidum and gonorrhea. U.S. Pat. No. 4,415,565, relevant portions incorporated herein be reference, of Wysor shows further antiviral activity of AgSD against certain RNA viruses, but none of these are retroviruses. Thus, while AgSD is a well studied material, there was no basis to expect that it would have activity against the AIDS retrovirus which has proven so difficult to inhibit or destroy.

According to B. Hanke, U.S. Pat. No. 6,720,006, relevant portions incorporated herein be reference, silver nanoparticles have demonstrated being useful to produce anti-microbial body care products. This opens the possibility of further studies in this area; however no antiviral testing was conducted.

In view of these findings, the invention contemplates a method of inhibiting the transmission of AIDS in humans upon sexual intercourse, by the use of an effective antiviral amount of silver nanoparticles topically applied to a sexual canal of a human prior to or during sexual intercourse. This application can be carried out by introducing a cream or foam into the sexual canal, or by coating the inhibitory composition onto a condom or other device that is inserted into the sexual canal.

There is a lack of studies analyzing the health impact of silver nanoparticles inside the human body. However, there is evidence that silver nanoparticles in proper concentrations are not dangerous for external use, U.S. Pat. No. 6,720,006, relevant portions incorporated herein be reference, and many references about the use of colloidal silver for health purposes.

There are several articles about the bactericidal properties of ionic silver. However, these articles focus on the known properties of silver nanoparticles (I. Sondi, B. Salopek-Sondi, J. Colloid Interface Sci. 275, 177-182 (2004) relevant portions, methods of manufacture and preparation, incorporated herein be reference) against bacteria.

Dosage Forms. The silver nanoparticles may also be administered, e.g., parenterally, intraperitoneally, intraspinally, intravenously, intramuscularly, intravaginally, subcutaneously, or intracerebrally. Dispersions may be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, poly-ol (for example, glycerol, propylene glycol, and liquid polyethylene glycol, and the like), suitable mixtures thereof, and vegetable oils.

The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Sterile injectable solutions may be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the therapeutic compound) plus any additional desired ingredient from a previously sterile-filtered solution thereof.

The silver nanoparticles may be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied as will be known to the skilled artisan. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a subject.

Aqueous compositions of the present invention comprise an effective amount of the nanoparticle, nanofibril or nanoshell or chemical composition of the present invention dissolved and/or dispersed in a pharmaceutically acceptable carrier and/or aqueous medium. The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of the nanoshell composition as an active component and/or ingredient will be known to those of skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts may be prepared in water suitably mixed with a surfactant, such as hydroxypropylcellulose. Dispersions may also be prepared in glycerol, liquid polyethylene glycols, and/or mixtures thereof and/or in oils. Under ordinary conditions of storage and/or use, these preparations contain a preservative to prevent the growth of microorganisms.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the required other ingredients from those enumerated above. In the case of sterile powders for the preparation of sterile injectable solutions, the preferred methods of preparation are vacuum-drying and/or freeze-drying techniques that yield a powder of the active ingredient plus any additional desired ingredient from a previously sterile-filtered solution thereof The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like may also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media that may be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

Additional formulations that are suitable for other modes of administration include vaginal suppositories and/or suppositories. A rectal suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

Substrates. The substrate of the compositions of the present invention may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A powder constitutes a finely divided (milled, micronized, nanosized, precipitated) form of an active ingredient or additive molecular aggregates or a compound aggregate of multiple components or a physical mixture of aggregates of an active ingredient and/or additives. Such substrates may be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapitite, tricalcium phosphate, talc, or titania; and polyols, such as mannitol, xylitol, sorbitol or cyclodextrin.

It should be emphasized that a substrate need not be a solid material, although often it will be a solid. For example, the encapsulation coat on the substrate may act as a solid “shell” surrounding and encapsulating a liquid, semi-liquid, powder or other substrate material. Such substrates are also within the scope of the present invention, as it is ultimately the carrier, of which the substrate is a part, which must be a solid.

Excipients. The silver nanoparticle pharmaceutical compositions of the present invention may include optionally one or more additives, sometimes referred to as additives. The excipients may be contained in an encapsulation coat in compositions, which include an encapsulation coat, or can be part of the solid carrier, such as coated to an encapsulation coat, or contained within the components forming the solid carrier. Alternatively, the excipients may be contained in the pharmaceutical composition but not part of the solid carrier itself. Suitable excipients are those used commonly to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, as are well known in the art.

The silver nanoparticles of the present invention may be used as a topical cream against HIV and other retroviruses. The cream described may also be used in condoms.

Sterile intravenous (iv) solution such as saline may be effective in reducing virus load and slowing down the onset of immunodeficiency. Surgeons who also use saline washes in cleansing a particular area in the operating field may find it useful. The silver nanoparticles may be used alone or in conjunction with a liposome. These forms could be reconstituted in the form of mouthwash with the silver nanoparticles alone or in conjunction with antifungal reagents. An inhalant form alone or in conjunction with pentamidine. The use of silver nanoparticles in tablet form to be taken orally. The oral use of the liposomal form would have to be given in a time release capsule to avoid lipase degradation.

Buffered ophthalmic solution—for patents suffering from HIV associated retinitis. The buffering is necessary due to pH changes the silver nanoparticles may cause. Highly concentrated solution for intramuscular injection—would facilitate treatment of needle stick injuries of health care workers. In this regard use of DMSO as solvent would give extremely fast penetration delivering high concentrations of silver nanoparticles to a small area.

Suppository form—for chemoprevention in homosexuals because the major sites of infection are the large intestine and rectum.

Chemo-preventative Vaginal douche and creme—the douche may be of use in a pre-sexual exposure in a standard acetic acid solution. The creme may be mixed with 9-nonoxynol spermicide to use in conjunction with birth control.

Vaginal sponge—this could be used by prostitutes so that silver nanoparticles would be time-released over several hours with nonoxynol.

Gloves lined with silver nanoparticles may help surgeons and other health care workers dealing heavily with blood and bodily fluids.

The use of silver nanoparticles in liquid soap in combination with anti-bacterial agents may be useful in hospitals and research institutions. Although this would probably be no more effective than plain anti-bacterial soap, the employees and hospital insurance companies would appreciate it.

Noble metal nanoparticle or nanowire-polyols or polymer complexes may be added slowly to an aqueous solution of polyvinylpyrrolidone and mixed well. Next, No. 25-30 mesh sugar spheres are coated with the noble metal nanoparticle or nanowire-polyols or polymer complex-drug solution in a fluid bed granulator. The drug containing pellets were dried, and a seal coat of Opadry Clear and the inner mixed release coating applied to the active particles by spraying a solution of ethylcellulose and diethyl phthalate in 98/2 acetone/water. The outer coating of a blend of ethylcellulose and HPMCP plasticized with diethyl phthalate was sprayed onto the active particles having the inner coating to produce modified release profile beads. These beads are filled into hard gelatin capsules using capsule filling equipment to produce noble metal nanoparticle or nanowire-polyols or polymer complex mini-tabs, 2.5, 5.0, 7.5, 8.0, 12.0, 16.0 and 20.0 mg.

A capsule for immediate release of a first active and extended release of a second active in an enveloped formulation, in a single capsule. The noble metal nanoparticle or nanowire-polyols or polymer complexes may be freeze-sprayed, lyophilized, vacuum dried, heat dried, heat-vacuum dried, etc. to form a powder following isolation and purification. The following is an example of the noble metal nanoparticle or nanowire-polyols or polymer complexes as part of a capsule. The skilled artisan will recognize that these formulations may be prepared in mixed immediate, intermediate and long-term or extended release.

Noble metal nanoparticle or nanowire-polyols or polymer complexes

| Talc | |
|---|---|
| Povidone K-30 | |
| Maltodextrin MD-40 | |
| Syloid | |
| Stearic Acid | |
| Capsule | 1 |

A formulation for release in a gelcap:

Noble metal nanoparticle or nanowire-polyols or polymer complexes

| Talc | |
|---|---|
| Povidone K-30 | |
| Maltodextrin MD-40 | |
| Syloid | |
| Stearic Acid | |
| Gelcap | 1 |

A formulation for release of the active in a suppository:

Noble metal nanoparticle or nanowire-polyols or polymer complexes

Talc

Povidone K-30

Maltodextrin MD-40

Syloid

Stearic Acid

beeswax/glycerol

An effervescent tablet for immediate release of a first active and extended release of a second active in an enveloped formulation, in an effervescent tablet:

Noble metal nanoparticle or nanowire-polyols or polymer complexes

Talc

Povidone K-30

Maltodextrin MD-40

Stearic Acid

Sodium bicarbonate

For immediate release in a caplet:

Noble metal nanoparticle or nanowire-polyols or polymer complexes

Talc

Povidone K-30

Maltodextrin MD-40

Stearic Acid

Compressed into a Caplet

In a liquid composition, the present invention may be provided as follows:

Noble metal nanoparticle or nanowire-polyols or polymer complexes

Excipient

Flavorant

Biocompatible Isotonic liquid (e.g., saline)

Buffer

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

(1) Burda, C.; Chen, X.; Narayanan, R.; El-Sayed, M. A. Chem. Rev. 2005, 105, 1025.
(2) Nam, J.-M.; Thaxton, C. S.; Mirkin, C. A. Science 2003, 301, 1884.
(3) Bonnemann, H.; Richards, R. M. Eur. J. Inorg. Chem. 2001, 10, 2455.
(4) Di Ventra, M.; Evoy, S.; Heflin, J. R. Introduction to nanoscale science and technology; Kluwer Academic Publishers: Boston, 2004.
(5) Liz-Marzan, L. M. Materials Today 2004, 7, 26.
(6) Mulvaney, P. Langmuir 1996, 12, 788.
(7) Xia, Y.; Halas, N. MRS Bulletin 2005, 30, 338.
(8) Schultz, S.; Smith, D. R.; Mock, J. J.; Schultz, D. A. PNAS 2000, 97, 996.
(9) Mock, J. J.; Barbic, M.; Smith, D. R.; Schultz, D. A.; Schultz, S. J. Chem. Phys. 2002, 116, 6755.
(10) Elechiguerra, J.; Burt, J. L.; Morones, J. R.; Camacho-Bragado, A.; Gao, X.; Lara, H. H.; Jose Yacaman, M. J. Nanobiotechnol. 2005, 3:6.
(11) Wiley, B.; Sun, Y.; Mayers, B.; Xia, Y. Chem-Eur. J. 2005, 11, 454.
(12) Yacaman, M. J.; Ascencio, J. A.; Liu, H. B.; Gardea-Torresdey, J. J. Vac. Sci. Technol. B 2001, 19, 1091.
(13) Doraiswamy, N.; Marks, L. D. Surf Sci. 1996, 348, 67.
(14) Iijima, S.; Ichihashi, T. Phys. Rev. Lett. 1986, 56, 616.
(15) Smith, D. J.; Petford-Long, A. K.; Wallenberg, L. R.; Bovin, J. O. Science 1986, 233, 872.
(16) Liu, H. B.; Ascencio, J. A.; Perez-Alvarez, M.; Yacaman, M. J. Surf. Sci. 2001, 491, 88.
(17) Lang, H.; Maldonado, S.; Stevenson, K. J.; Chandler, B. D. J. Am. Chem. Soc. 2004, 126, 12949.
(18) Sun, Y.; Xia, Y. Science 2002, 298, 2176.
(19) Sun, Y.; Xia, Y. Adv. Mater. 2002, 14, 833.
(20) Jana, N. R.; Gearheart, L.; Murphy, C. J. Adv. Mater. 2001, 13, 1389.
(21) Kim, F.; Connor, S.; Song, H.; Kuykendall, T.; Yang, P. Angew. Chem. Int. Ed. 2004, 43, 3673.
(22) Bonet, F.; Guery, C.; Guyomard, D.; Urbina, R. H.; Tekaia-Elhsissen, K.; Tarascon, J. M. Int. J. Inor. Mater. 1999, 1, 47.
(23) Gao, Y.; Jiang, P.; Liu, D. F.; Yuan, H. J.; Yan, X. Q.; Zhou, Z. P.; Wang, J. X.; Song, L.; Liu, L. F.; Zhou, W. Y.; Wang, G.; Wang, C. Y.; Xie, S. S.; Zhang, J. M.; Shen, D. Y. J. Phys. Chem. B 2004, 108, 12877.
(24) Bennett, H. E.; Peck, R. L.; Burge, D. K.; Bennett, J. M. J. Appl. Phys. 1969, 40, 3351.
(25) Franey, J. P.; Kammlott, G. W.; Graedel, T. E. Corros. Sci. 1985, 25, 133.
(26) Graedel, T. E.; Franey, J. P.; Gualtieri, G. J.; Kammlott, G. W.; Malm, D. L. Corros. Sci. 1985, 25, 1163.
(27) Volpe, L.; Peterson, P. J. Corros. Sci. 1989, 29, 1179.
(28) Graedel, T. E. J. Electrochem. Soc. 1992, 139, 1963.
(29) Chen, H.; Gao, Y.; Zhang, H.; Liu, L.; Yu, H.; Tian, H.; Xie, S.; Li, J. J. Phys. Chem. B 2004, 108, 12038.
(30) Sun, Y.; Mayers, B.; Herricks, T.; Xia, Y. Nano Lett. 2003, 3, 955.
(31) Huang, H. H.; Ni, X. P.; Loy, G. L.; Chew, C. H.; Tan, K. L.; Loh, F. C.; Deng, J. F.; Xu, G. Q. Langmuir 1996, 12, 909.
(32) Moulder, J. F.; Chastain, J.; King, R. C. Handbook of x-ray photoelectron spectroscopy: a reference book of standard spectra for identification and interpretation of XPS data; Physical Electronics: Eden Prairie, Minn., 1995.
(33) Reyes-Gasga, J.; R. Garcia G.; Jose-Yacaman, M. Rad. Phys. Chem. 1995, 45, 283.
(34) Allpress, J. G.; Sanders, J. V. Philos. Mag. 1966, 13, 609.
(35) Ino, S.; Ogawa, S. J Phys. Soc. Jap. 1967, 22, 1365.
(36) Marks, L. D.; Smith, D. J. J. Cryst. Growth 1981, 54, 425.
(37) Marks, L. D. Philos. Mag A 1984, 49, 81.
(38) Heinemann, K.; Yacaman, M. J.; Yang, C. Y.; Poppa, H. J. Cryst. Growth 1979, 47, 177.
(39) Howie, A.; Marks, L. D. Philos. Mag. A 1984, 49, 95.
(40) Chen, H.; Gao, Y.; Yu, H.; Zhang, H.; Liu, L.; Shi, Y.; Tian, H.; Xie, S.; Li, J. Micron 2004, 35, 469-474.
(41) Hyuk, S. I.; Lee, Y. T.; Wiley, B.; Xia, Y. Angew. Chem. Inter. Ed. 2005, 44, 2154.
(42) Allpress, J. G.; Sanders, J. V. Philos. Mag. 1964, 10, 827.
(43) Schloetterer, H. Phys. Status Solidi 1965, 11, 219.
(44) Forty, A. J.; Frank, F. C. Proc. Roy. Soc. 1953, A217, 262.
(45) Chang, R. K.; Furtak, T. E. Surface Enhanced Raman Scaterring; Plenum Press: New York, 1982.
(46) Kneipp, K.; Kneipp, H.; Itzkan, I.; Dasari, R. R.; Feld, M. S. Chem. Rev. 1999, 99, 2957
(47) Kreibig U.; Vollmer M. Optical Properties of Metal Clusters; Springer-Verlag: Heidelberg, 1995.
(48) Haynes, C. L.; Yonzon, C. R.; Zhang, X.; Van Duyne, R. P. J. Raman. Spectrosc. 2005, 36, 471.
(49) Daniels, V; Studies in Conservation 1981, 26,45.
(50) Kitova, S.; Eneva, J.; Panov, A.; Haefke, H. J. Imaging Sci. Techn. 1994, 38, 484.

What is claimed is:

1. An anti-viral composition comprising one or more polyol-nanoparticles.

2. The composition of claim 1, wherein the silver nanoparticles comprises a nanowire.

3. The composition of claim 1, wherein the silver nanoparticles comprises a nanowire comprising a diameter of between about 1 and 100 nm.

4. The composition of claim 1, wherein the silver nanoparticles comprises a nanowire comprising a length of between about 10 and 1,000 nm.

5. The composition of claim 1, wherein the silver nanoparticles comprises a nanowire provided at a concentration of at least about 3 μg/mL or greater.

6. The composition of claim 1, further comprising a capping agent.

7. The composition of claim 1, wherein the nanoparticles are polyol capped.

8. The composition of claim 1, wherein the nanoparticles are capped with a Poly(vinylpyrrolidone) or a poly(diallyldimethyl ammonium chloride).

9. A biocidal composition comprising one or more nanoparticles selected from the group consisting of gold, platinum, palladium, copper, iron, and alloys thereof.

10. The composition of claim 9, wherein the nanoparticles comprise a nanowire.

11. The composition of claim 9, wherein the nanoparticles comprise a nanowire comprising a diameter of between about 1 and 100 nm.

12. The composition of claim 9, wherein the polyol comprises one or more of the following: alkylene glycols (e.g., 1,2-ethanediol, 1,2-propanediol, 3-chloro-1,2-propanediol, 1,3-propanediol, 1,3-butanediol, 1,4-butanediol, 2-methyl-1,3-propanediol, 2,2-dimethyl-1,3-propanediol (neopentylglycol), 2-ethyl-1,3-propanediol, 2,2-diethyl-1,3-propanediol, 1,5-pentanediol, 2-ethyl-1,3-pentanediol, 2,2,4-trimethyl-1,3-pentanediol, 3-methyl-1,5-pentanediol, 1,2-, 1,5-, and 1,6-hexanediol, 2-ethyl- 1,6-hexanediol, bis(hydroxymethyl)cyclohexane, 1,8-octanediol, bicyclo-octanediol, 1,10-decanediol, tricyclo-decanediol, norbornanediol, and 1,18-dihydroxyoctadecane); polyhydroxyalkanes (e.g., glycerine, trimethylolethane, trimethylolpropane, 2-ethyl-2-(hydroxymethyl)-1,3-propanediol, 1,2,6-hexanetriol, pentaerythritol, quinitol, mannitol, and sorbitol); and other polyhydroxy compounds, e.g., diethylene glycol, triethylene glycol, tetraethylene glycol, tetramethylene glycol, dipropylene glycol, diisopropylene glycol, tripropylene glycol, 1,11-(3,6-dioxaundecane)diol, 1,14-(3,6,9,12-tetraoxatetradecane)diol, 1,8-(3,6-dioxa-2,5,8-trimethyloctane)diol, 1,14-(5,10-dioxatetradecane)diol, castor oil, 2-butyne-1,4-diol, N,N-bis(hydroxyethyl)benzamide, 4,4′-bis(hydroxymethyl)diphenylsulfone, 1,4-benzenedimethanol, 1,3-bis(2-hydroxyethyoxy)benzene, 1,2-, 1,3-, and 1,4-resorcinol, 1,6-, 2,6-, 2,5-, and 2,7-dihydroxynaphthalene, 2,2′- and 4,4′-biphenol, 1,8-dihydroxybiphenyl, 2,4-dihydroxy-6-methyl-pyrimidine, 4,6-dihydroxypyrimidine, 3,6-dihydroxypyridazine, bisphenol A, 4,4′-ethylidenebisphenol, 4,4′-isopropylidenebis(2,6-dimethylphenol), bis(4-hydroxyphenyl)methane, 1,1-bis(4-hydroxyphenyl)- 1-phenylethane (bisphenol C), 1,4-bis(2-hydroxyethyl)piperazine, bis(4-hydroxyphenyl) ether, as well as other aliphatic, heteroaliphatic, saturated alicyclic, aromatic, saturated heteroalicyclic, and heteroaromatic polyols, polymeric polyols such as polyoxyethylene, polyoxypropylene and ethylene oxide-terminated polypropylene glycols and triols; polytetramethylene glycols; polydialkylsiloxane diols; hydroxy-terminated polyesters and hydroxy-terminated polylactones (e.g., polycaprolactone polyols); hydroxy-terminated polyalkadienes (e.g., hydroxyl-terminated polybutadienes) combinations or mixtures thereof.

13. The composition of claim 9, wherein the nanoparticles comprise a nanowire provided at a concentration of at least about 3 μg/mL or greater.

14. The composition of claim 9, wherein the silver nanoparticles are made available in a solution, suspension, cream, ointment, lotion, enema, elixir, syrup, emulsion, gum, gel, insert, suppository, jelly, foam, paste, pastille, spray, magma or poultice.

15. The composition of claim 9, wherein the silver nanoparticles are packaged for immediate release.

16. The composition of claim 9, wherein the silver nanoparticles are packaged for extended release.

17. The composition of claim 9, wherein the silver nanoparticles are enveloped in a single dose.

18. The composition of claim 9, wherein the silver nanoparticles are disposed in or about a condom.

19. The composition of claim 9, wherein the silver nanoparticles are packed into a capsule, caplet, softgel, gelcap, suppository, film, granule, gum, insert, pastille, pellet, troche, lozenge, disk, poultice or wafer.

20. The composition of claim 9, wherein over 80% of the silver nanoparticles are released within about 60 minutes.

21. The composition of claim 9, wherein the silver nanoparticles are provided for immediate release which comprises release of over 90% of the silver nanoparticles within about 90 minutes.

22. The composition of claim 9, wherein silver nanoparticles are packaged for extended release comprising release of over 80% of the silver nanoparticles within about 60 minutes to about 8 hours.

23. A method for preventing anti-viral infections comprising the steps of:
resuspending in a pharmaceutically acceptable carrier one or more nanoparticles dissolved and reduced with a polyol to form an anti-viral composition; and
isolating the nanoparticles.

24. The method of claims 23, further comprising the step of providing the nanoparticles to a mammal.

25. A method of treating a patient suspected of having a viral infection comprising the steps of:
providing a patient suspected of being in need of anti-viral therapy with a composition comprising nanoparticles dissolved and reduced with a polyol in a pharmaceutical acceptable carrier.

26. A method of claim 25, wherein the one or more nanoparticles are selected from the group consisting of silver, gold, platinum, palladium, copper, iron, and alloys thereof.
